(19) United States
(12) Patent Application Publication
(10) Pub. No.: US 2024/0139498 A1
(43) Pub. Date: May 2, 2024

(54) Assessment and Management of Adverse Event Risks in Mechanical Circulatory Support Patients

(71) Applicant: TC1 LLC, St. Paul, MN (US)

(72) Inventors: Rahul Agarwal, Venhura, CA (US); Rupinder Bharmi, Canyon Country, CA (US); Robert L. Kormos, Austin, TX (US); Yajing Hu, San Jose, CA (US); Qianhul Lu, Santa Clara, CA (US); Palak Shah, McLean, VA (US)

(73) Assignee: TC1 LLC, St. Paul, MN (US)

(21) Appl. No.: 18/498,765

(22) Filed: Oct. 31, 2023

Related U.S. Application Data
(60) Provisional application No. 63/421,450, filed on Nov. 1, 2022.

(51) Int. Cl. A61M 60/178; G16H 50/30

(52) U.S. Cl. CPC A61M 60/178; G16H 50/30; A61M 2205/18

(57) ABSTRACT

Systems and methods for assessment and management of adverse event risks in mechanical circulatory support patients are described herein. The method for post-operative risk mitigation in patients with an implanted Ventricular Assist Device (VAD) can include receiving a plurality of features relating to an attribute associated with the patient and ingesting at least some of the features into a multistate model. The multistate model can include a plurality of states, each corresponding to a patient condition. The method can include generating with the multistate model a daily prediction of a likelihood of the patient developing at least one of the conditions corresponding to the plurality of states in a predetermined time period, and controlling a user interface to output an indicator of the likelihood of the patient developing the at least one of the conditions in the predetermined time period.

### CROSS REFERENCE TO RELATED APPLICATION DATA

The present application claims the benefit under 35 USC § 119(e) of U.S. Provisional Appln. No. 63/421,450 filed Nov. 1, 2022; the full disclosure which is incorporated herein by reference in its entirety for all purposes.

### BACKGROUND

Ventricular assist devices, known as VADs, are implantable blood pumps used for both short-term (i.e., days, months) and long-term applications (i.e., years or a lifetime) where a patient's heart is incapable of providing adequate circulation, commonly referred to as heart failure or congestive heart failure. According to the American Heart Association, more than six million Americans are living with heart failure, with about 670,000 new cases diagnosed every year. People with heart failure often have shortness of breath and fatigue. Years of living with blocked arteries or high blood pressure can leave a heart too weak to pump enough blood to the body. As symptoms worsen, advanced heart failure develops.

A patient suffering from heart failure, also called congestive heart failure, may use a VAD while awaiting a heart transplant or as a long-term destination therapy. In another example, a patient may use a VAD while their own native heart recovers. Thus, a VAD can supplement a weak heart (i.e., partial support) or can effectively replace the natural heart's function. VADs can be implanted in the patient's body and powered by an electrical power source inside or outside the patient's body.

### BRIEF SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

It is estimated that approximately 6.2 million adults in the United States live with some level of heart failure, and heart failure was listed as the cause of death on nearly 380,000 U.S. death certificates in 2018. Heart failure can be treated in many different ways, including via medications, surgical repair of heart tissue, or via implantation of medical device such as a ventricular assist device (VAD).

Continuous-flow left ventricular assist devices (LVAD) have been proven to be an effective therapy for some patients with advanced heart failure. However, certain risks associated with use of an LVAD have prevented widespread adoption of this therapy. While one and two-year survival continue to improve, the incidence of gastrointestinal bleeding (GIB) and stroke remain high after implantation of an LVAD.

Contemporary LVAD patient management focuses on correctively addressing adverse events as they arise, and not on prognosticating patients for risk of bleeding or thromboembolic event and then treating based on that prognostication.

In many embodiments, an automated patient risk estimator can predict impending adverse events. This can include and/or result in the adjusting of patient management strategies to intercede in the chain of events to prevent this impending adverse event, including adjusting therapeutic plans based on a risk or risks determined by the patient risk estimator.

The patient risk estimator can calculate risk pre-operatively, can predict risk during operation, or can predict risk at different times post-operatively. These risk predictions can improve decision making with respect to whether and/or which therapies to provide. These risk predictions can be general, in that they predict the risk of the occurrence of any adverse event, or can be specific in that they predict the risk of the occurrence of one or several specific adverse events.

Based on these risk predictions, patient care can be planned or changed, which can include the creation of treatment plans for one or several high-risk patients. Further based on these risk predictions and significance of features, one or several modifiable risk factors can be identified and a treatment plan to mitigate those one or several factors can be created and/or provided. Further, based on the risk score and/or a change in risk score, patients can be triaged such that patient with higher risk scores and/or adverse changes in risk score are prioritized for care or checkups, or such that lower risk patients are identified for remote clinical management. Through the generation of multiple risk predictions for a patient over time, the clinical trajectory of the patient can be determined, which clinical trajectory can serve as an input for further risk predictions.

In some embodiments, a patient risk of positive and/or adverse outcomes is predicted with a machine learning model. This prediction can include a prediction of a general adverse outcome, or a prediction of one or several specific adverse outcomes, such as, for example, risk of gastrointestinal bleeding, risk of stroke, risk of infection, risk of death, and/or risk of arrythmia.

The machine learning model can be configured to generate predictions of different patient outcomes. This machine learning model can be a multistate model that can predict the likelihood of multiple outcomes.

This prediction can be regularly and/or continuously updated as time passes and/or as new information relating to the patient is received. In some embodiments, the generation of regular and/or of continuous predictions can include the generation of data, which can be used as features to enable such a prediction. For example, a prediction can be generated without the benefit of having a completely new set of data for use in generating this prediction. In such embodiments, data can be generated for use in generating the prediction. This data can be generated via, for example, identifying a cohort of patients similar to the patient for whom the data is missing, and from this cohort, extracting data to resolve the data insufficiency. In some embodiments, this data can be generated based on information such as, for example, a medication decay projection, extrapolation, a most recent value, or the like.

In some embodiments, and based on this prediction, control of the LVAD can be affected. For example, one or several of the parameters of the LVAD can be automatically adjusted. This can include, for example, adjusting the pump speed and specifically adjusting a mean rotational speed of the pump. This adjustment can be to target the risk of the specific predicted adverse event.

Through the use of the patient risk predictor, patient care and outcomes can be improved. A patient trending towards an adverse outcome can be identified, and targeted, preventive care can be delivered to this patient before the adverse event is manifest. Such proactive care can increase the effectiveness of the LVADs and improve the ability of a doctor to treat heart failure.

For a fuller understanding of the nature and advantages of the present invention, reference should be made to the ensuing detailed description and accompanying drawings.

### BRIEF DESCRIPTION OF THE DRAWINGS

FIG. 1 is an illustration of a mechanical circulatory support system that includes a ventricular assist device (VAD) implanted in a patient's body, in accordance with many embodiments.

FIG. 2 is an exploded view of implanted components of the circulatory support system of FIG. 1.

FIG. 3 is an illustration of the VAD of FIG. 1 attached to the patient's heart to augment blood pumping by the patient's left ventricle.

FIG. 4 is an illustration of a mechanical circulatory support system that includes implantable transcutaneous energy transmission system receiver, an implantable controller, and a ventricular assist device (VAD), in accordance with many embodiments.

FIG. 5 is an illustration of a blood circulatory support system that includes the VAD implanted in a patient's body, in accordance with many embodiments.

FIG. 6 is a schematic depiction of one embodiment of the controller of the mechanical circulatory support system.

FIG. 7 is a schematic depiction of one embodiment of the multistate model.

FIG. 8 is a flowchart illustrating one embodiment of a process for post-operative risk mitigation in patients with an implanted Ventricular Assist Device (VAD).

FIG. 9 is a flowchart illustrating one embodiment of a process for generating replacement feature.

FIG. 10 is a flowchart illustrating one embodiment of a process for generating replacement feature based on a proxy cohort or proxy patient.

FIG. 11 is a flowchart illustrating one embodiment of a process for generating replacement feature via scheduling of an action.

FIG. 12 is graphical prediction of observed GI bleeding by risk group.

FIG. 13 is graphical prediction of observed death by risk group.

FIG. 14 is graphical prediction of observed stroke by risk group.

FIG. 15 is a graphical prediction of observed adverse events by risk group.

FIG. 16 depicts features associated with GI bleeding.

FIG. 17 depicts features associated with mortality.

FIG. 18 depicts features associated with stroke.

FIG. 19 shows model performance at any day after discharge after a VAD implant using the 5-fold cross-validation on derivation data.

FIG. 20 shows model performance at any day after discharge after a VAD implant using the validation dataset.

### DETAILED DESCRIPTION

In the following description, various embodiments of the present invention will be described. For purposes of explanation, specific configurations and details are set forth in order to provide a thorough understanding of the embodiments. However, it will also be apparent to one skilled in the art that the present invention may be practiced without the specific details.

Furthermore, well-known features may be omitted or simplified in order not to obscure the embodiment being described.

### Patient Risk Model

It is estimated that approximately 6.2 million adults in the United States live with some level heart failure, and heart failure was listed as the cause of death on nearly 380,000 U.S. death certificates in 2018. Heart failure can be treated in many different ways, including via medications, surgical repair of heart tissue, or via implantation of medical device such as a ventricular assist device (VAD).

Continuous-flow left ventricular assist devices (LVAD) have been proven to be an effective therapy for some patients with advanced heart failure. However, as with any surgical intervention, certain risks are associated with use of an LVAD. While one and two-year survival continue to improve, the incidence of gastrointestinal bleeding (GIB) and stroke remain higher than desired one-year after implantation of an LVAD.

Contemporary LVAD patient management focuses on correctively addressing adverse events as they arise. Contemporary treatment does not prognosticate patients for risk of bleeding or thromboembolic event and then treat based on that prognostication. The present is directed at systems and methods for shifting LVAD patient management from reactive treatment as adverse event arise to proactive treatment that addresses risks and changing risks of adverse events.

A patient risk estimator can enable proactive treatment to address risks and changing risks of adverse events by utilizing patient data to generate a risk prediction for the patient on a frequent, and more specifically on a daily basis. This risk prediction can, in some embodiments, be a real-time risk prediction and/or a dynamic risk prediction. The patient risk estimator utilizes a machine learning model specifically configured to manage the multiple potential adverse events in the form of multiple states. These states are each interlinked to other states within a multistate model. In contrast to other models, the patient risk estimator can generate frequent, including daily, risk predictions which can include a patient frequently transitioning state. This state transition can, for example, be up to on a daily basis, or can be even more frequent. This high frequency of state transitions can be achieved via a multistate model that models daily transition probabilities given current state, which are estimated by logistic regression models.

The use of the logistic regression multistate model provides several benefits. Specifically, the multistate model allows risk predictions on a daily basis. Further, the logistic regression for each state transition provides interpretability of feature weights and odds ratios.

For the multistate model, let X(t)∈{0,1}ⁿ be a state vector representing probability of being in a state I at time t.

Then the multistate model assumes:

X(t+1)=P(t, θ(t))X(t)

Where P (t, θ(t))∈R^{n×n} is state transition matrix. Which gives daily probability of transition from a current state to next.

In these equations, θ(t) represents the patient specific covariate, that may also change over time, such as lab values, demographics, pump parameters etc.

For calculating transition over multiple days, d, MSM allows use of following relation:

$$X ( t + d ) = ( {\prod }_{τ = t}^{t + d} P ( τ , θ ( τ ) ) ) X ( t )$$

The patient risk generator can be further configured to manage data to improve accuracy of risk predictions. This can include evaluating data, also referred to herein as features to determine if/when data is aged, referred to herein as aged data or aged features. The patient risk generate can access one or several rules to determine how to handle any aged data. This handling of any aged data can include the generation of replacement data, also referred to herein as replacement features, which can be used in the place of the aged data to generate the risk prediction. The replacement data can be generated according to one or several data replacement models.

The patient risk generator can further output the risk prediction via a user interface. This can include outputting individual risk predictions, or outputting a time-varying risk prediction in, for example, the form of a trendline. For example, as multiple risk predictions are generated over a period of time, these predictions can be provided to the user in the form of a time-varying trendline.

Individual risk predictions can be compared to one or several threshold values to determine when to trigger an alert and/or a risk mitigating action in response to the individual risk prediction. The trendline of multiple risk predictions can be evaluated to determine when the slope of the trendline changes, and whether that change indicates increase or decreasing risk, In some embodiments, the patient risk predictor can trigger an alert and/or recommend or take a risk mitigating action in response to a change in the slope of the trendline, and specifically when the change indicates increasing risk. In some embodiments, the patient risk predictor can trigger an alert and/or recommend or take a risk mitigating action when the slope of the trendline indicates risk increasing at or above a threshold value.

The patient risk estimator can predict impending adverse events via a multistate model that generates frequent risk predictions for individual patients, and in some instances, daily risk predictions for individual patients. These risk predictions can be generated based on patient information, also referred to herein as patient features or patient parameters, that can be gathered pre-implantation, during implantation (peri-implantation), and/or post implantation. The pre-implantation features can include, in some embodiments, baseline characteristics including demographics, comorbidities, medications, valvular disease status, or the like. In some embodiments, exemplary pre-implantation features can include, for example, patient demographics including age, neurological history, seizure history, stroke history, mean pulmonary arterial pressure, significant peripheral vascular disease history, whether the patient receive(d) epinephrine, transient ischemic attack, prior valve replacement or repair, gastrointestinal history, left ventricular end-diastolic diameter (LVEDD), left ventricular internal diameter end diastole and end systole (LVIDD), and/or heart failure duration.

The peri-implantation features can include, in some embodiments, implant characteristics such as, for example, hemodynamics and/or invasive hemodynamics, labs and/or lab examinations, procedural/surgical parameters, and pump parameters. The peri-implantation features can include, in some embodiments, events during implantation such a gastrointestinal bleeding (GI bleeding), other bleeding, cardiac arrhythmias, right heart failure, stroke, renal dysfunction, respiratory failure, and/or major infection. In some embodiments, specific features for a patient during implantation can include, for example, in hospital respiratory failure, and/or in hospital gastrointestinal bleeding.

The post-implantation features can include, in some embodiments, one or several

longitudinal variables and/or events during follow-up. The longitudinal variables can include, for example, lab results and/or lab examinations, medications and/or medication changes, pump parameters, vitals, duration of follow-up, and/or the existence of an adverse event after hospital discharge. In some embodiments, embodiments during follow-up can include, for example, major infections, right heart failure, and/or heart failure hospitalization. In some embodiments, specific post-implantation features for a patient can include, aspirin dose (low dose (<=100 mg), high dose (>100 mg), no dose), BUN (mg/dL), hemoglobin (g/dL), platelets (x10^3/uL), NTProBNP (pg/mL), total protein, pump pulsatility index, right heart failure, infection, heart failure hospitalization, pump speed, diuretic medications, albumin (g/dl), pump power, International Normalized Ratio (“INR”), and/or aspartate transferase (“AST”) (U/L). In some instances, this information can be routinely updated, which updates can affect the risk predictions.

A large set of variables was evaluated to select features for use in the model. Initially, each variable was evaluated for each state transition in a univariate analysis, and variables having a p-value>0.2 were removed. The remaining variables were used in multivariate analysis for each transition and any variables having p-value>0.2 were removed. Finally, variables from the multivariate model step were selected with 9 different p-value thresholds for each subsequent 5-fold cross validation. The final machine learning model for each outcome was built based on AUC or log likelihood optimized variables from the 9 cross validation results.

Data used in generated the risk predictions can be fixed data, or can be time variable. For example, pre-implantation patient data, and patient data during implantation are fixed once their associated time period has passed. In contrast to this, post-implantation patient data can vary with time. For example, the pre-operative patient weight cannot change from its final value once the operation begins. Similarly, data indicating whether an event, such as bleeding, took place during implantation is fixed once the implantation of the VAD is complete. In contrast to this, a post-operative patient weight will vary with time.

Data that is time variable, can age. For example, if a patient weight is used to generate a risk prediction, use of an old, and no longer accurate post-operative patient weight may not yield an accurate risk prediction. Depending on the frequency of information updates versus the frequency of risk predictions, a risk prediction may be generated with aged data. Aged data, as used herein, is time variable data that is older than a predetermined time period and/or that has been previously used in generation of a risk prediction.

When it is determined to generate a risk prediction, data for the generation of the risk prediction can be gathered and can be evaluated to identify if any, some, or all of the data is aged. If some of the data is aged, then the aged data can be evaluated to determined attributes of the aged data including, for example, one or several rules for handling of the aged data. In some embodiments, for example, depending on the type of aged feature, and/or the degree to which the feature has aged, some aged data may still be used in generation of a risk prediction, whereas other aged data may be replaced via replacement data.

The replacement data can be gathered, created, and/or selected by the patient risk estimator. This can include, for example, automatically scheduling an action that will result in the creation and/or gathering of new data, such as, for example, the automatic scheduling of an appointment, a treatment, or the like. Generation and/or creation of replacement data can further be according to a data replacement model, which models can include, for example, a proxy model in which replacement data is generated and/or created based on a pool of one or more patients identified as a proxy to the current patient.

Features used in generating a patient risk prediction can include some or all of the replacement features and/or from some or all of the aged features and/or non-aged features. The features can be pieces of data that can be input into the multistate model, also referred to herein as ingested into the multistate model, to generate the risk prediction.

Some or all of these features can be ingested by the multistate model, which model can generate one or several risk predictions. In some embodiments, this multistate model can comprise a first plurality of interconnected states, and can generate a prediction of a likelihood of the patient having an adverse event associated with one or more of a second plurality of states. This second plurality of states can be less than the first plurality of states.

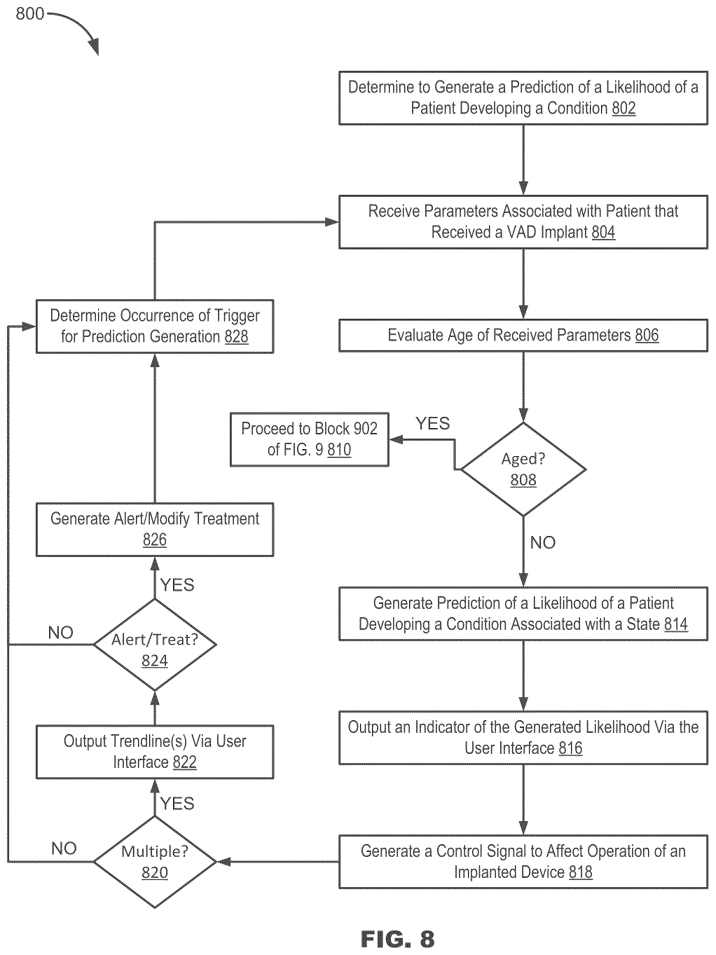

The multistate model can generate regular and/or frequent risk predictions. These risk predictions can be for an individual patient. In some embodiments, these risk predictions can be generated by the patient risk estimator on a daily basis. Thus, in some embodiments, a patient can remain in one state or progress to the next state as illustrated in FIG. 7 in the multistate model on a daily basis. For example, the patient's state in the model corresponds to their physical state. Thus, a patient that is alive and has not had an event will be in the alive/no event state. If that same patient develops a gastrointestinal bleed, they will be advanced to the GI bleed state. Once a patient has transitioned to a new state, they remain in that new state unless the patient experiences a subsequent state transition.

The multistate model can be specialized for frequent, and specifically for up to daily, state transitions. This can include, for example, utilizing logistic regression in the multistate model to estimate transition probability. Thus, in some embodiments, the multistate model can be a logistic regression multistate model.

The automated patient risk predictor can successfully predict a patient's risk of experiencing an adverse event. Via this successful prediction, a care provider can take steps to pre-emptively address and/or mitigate the risk of the occurrence of the adverse event. Through such treatments, patient outcomes are improved.

### Circulatory Support System

In many embodiments described herein, a circulatory support system includes a ventricular assist device (VAD) for a patient, electrocardiogram electrodes that are used to monitor cardiac activity of the patient, and a controller that controls operation of the VAD based on the cardiac activity of the patient. For example, the mean rotational speed of the VAD can be varied based on an activity level of the patient determined by the controller based on the cardiac activity of the patient as measured by the electrocardiogram electrodes. As another example, the VAD can be operated in a pulsatile mode that produces a blood pressure pulse (via an increase in the rotational speed of the VAD) that is synchronized with a cardiac cycle of the patient.

Many existing ventricular assist devices essentially operate in constant speed mode in which the VAD rotor rotation rate is held constant and set by a clinician before patient discharge. Both centrifugal and axial flow pumps exhibit unique inverse relationships between flow and pressure at every speed as illustrated in a head-flow (HQ) curve for the pump. For a left ventricular assist device (LVAD), the average flow through the LVAD does increase somewhat in response to a drop in aortic pressure, which typically occurs as a result of an increase of the activity level of the patient. The increase in average flow through the LVAD produced by the reduced aortic pressure, however, is small compared to the native heart's ability to adjust cardiac output between rest and exercise states (Native ˜4-25 L/min, VAD ˜3-6 L/min).

In many embodiments described herein, a controller controls operation of a VAD to automatically adjust the mean rotational speed of the VAD in real time in response to a significant change in physiologic demand. For example, in many embodiments the controller reduces the mean rotational speed of the VAD during sleep or rest to prevent ventricular suction events, thereby enabling the means rotational speed of the VAD to be set higher to provide enhanced profusion/support without fear of encountering suction events during sleep or rest. In many embodiments, the controller increases the mean rotational speed of the VAD when the patient is in a more active state (exercise response).

Aortic insufficiency is another notable adverse event that influences the rotational speed selected by the clinician for a VAD operated at a constant rotational speed. Aortic insufficiency can be caused by structural failure of the aortic valve after chronic support. It is hypothesized that lack of physiologic cycling of the aortic valve during prolonged closure of the aortic valve causes biomechanical deterioration and subsequent prolapse of the aortic valve. In an attempt to avoid the occurrence of aortic insufficiency, a clinician may set the rotational speed of the VAD to achieving periodic aortic valve opening rather than optimal hemodynamic support. In some embodiments, a controller controls operation of a VAD to periodically reduces the rotational speed of the VAD (at least during ventricular systole) to induce opening and closing of the aortic valve.

In some embodiments described herein, a controller controls operation of a VAD to operate in an artificial pulse mode that is synchronized with the cardiac cycle of the patient. In some existing circulatory support systems, a VAD is operated in an asynchronous manner in which the rotational speed of the VAD is varied in a repeated pattern on a fixed cycle time basis (e.g., one cycle every two seconds) regardless of the native cardiac cycle timing. While operation of a VAD in an asynchronous artificial pulse mode may provide several clinical benefits, such as inhibiting thrombus formation via enhanced washing of the VAD, the left ventricle, the aortic root, inhibiting the development of aortic insufficiency, operation of the VAD in an artificial pulse mode that is synchronized with the cardiac cycle may achieve improved aortic pulse pressure, improved aortic valve opening/cycling, improved ventricle unloading and wall cycling (which may be beneficial when used in some cardiac recovery approaches), and/or enhanced washing of the ventricle, the VAD, and/or the aortic root.

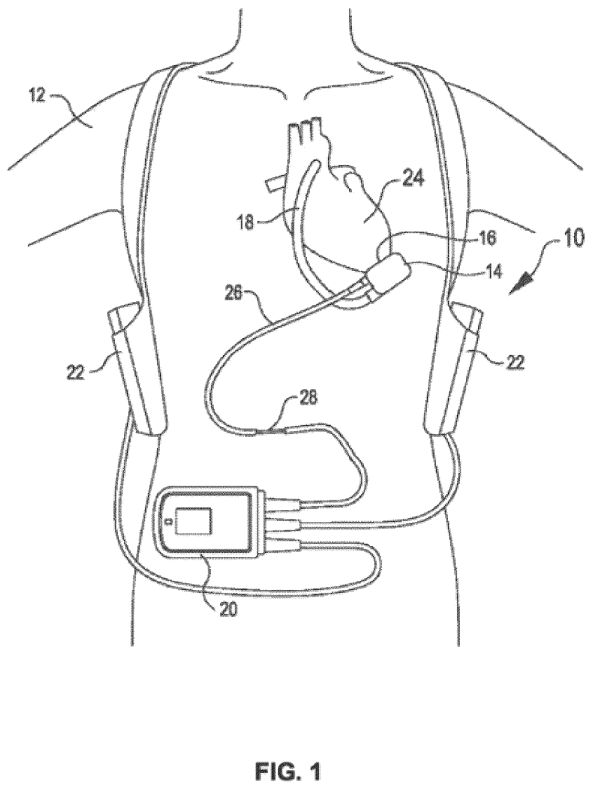

Referring now to the drawings, in which like reference numerals represent like parts throughout the several views, FIG. 1 is an illustration of a mechanical circulatory support system 10 that includes a ventricular assist device (VAD) 14 implanted in a patient's body 12. The mechanical circulatory support system 10 includes the VAD 14, a ventricular cuff 16, an outflow cannula 18, an external system controller 20, and power sources 22. A VAD 14 can be attached to an apex of the left ventricle, as illustrated, or the right ventricle, or a separate VAD can be attached to each of the ventricles of the heart 30. The VAD 14 can be capable of pumping the entire flow of blood delivered to the left ventricle from the pulmonary circulation (i.e., up to 10 liters per minute). Related blood pumps applicable to the present invention are described in greater detail below and in U.S. Pat. Nos. 5,695,471, 6,071,093, 6,116,862, 6,186,665, 6,234,772, 6,264,635, 6,688,861, 7,699,586, 7,976,271, 7,997,854, 8,007,254, 8,152,493, 8,419,609, 8,652,024, 8,668,473, 8,852,072, 8,864,643, 8,882,744, 9,068,572, 9,091,271, 9,265,870, and 9,382,908, all of which are incorporated herein by reference for all purposes in their entirety.

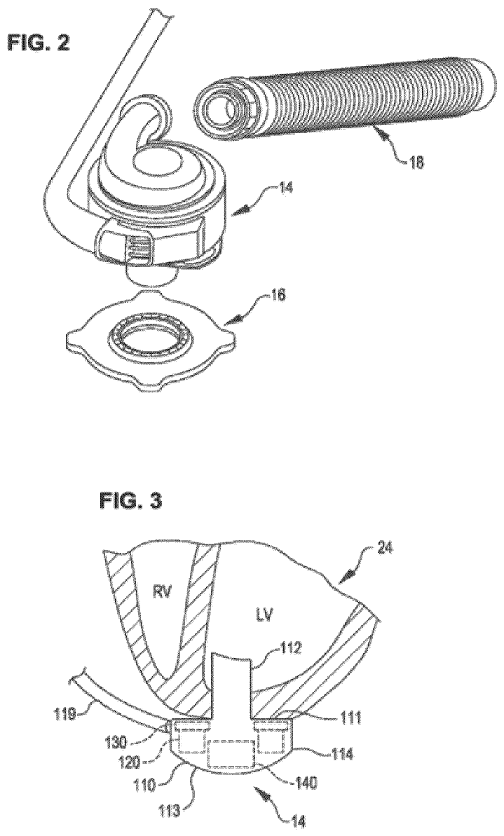

With reference to FIG. 1 and FIG. 2, the VAD 14 can be attached to the heart 30 via the ventricular cuff 16, which can be sewn to the heart 30 and coupled to the VAD 14. In the illustrated embodiment, the output of the VAD 14 connects to the ascending aorta via the outflow cannula 18 so that the VAD 14 effectively diverts blood from the left ventricle and propels it to the aorta for circulation through the rest of the patient's vascular system.

FIG. 1 illustrates the mechanical circulatory support system 10 during battery 22 powered operation. A driveline 26 that exits through the patient's abdomen 28 connects the VAD 14 to the external system controller 20, which monitors system 10 operation. Related controller systems applicable to the present invention are described in greater detail below and in U.S. Pat. Nos. 5,888,242, 6,991,595, 8,323,174, 8,449,444, 8,506,471, 8,597,350, and 8,657,733, EP 1812094, and U.S. Patent Publication Nos. 2005/0071001 and 2013/0314047, all of which are incorporated herein by reference for all purposes in their entirety. The system 10 can be powered by either one, two, or more batteries 22. It will be appreciated that although the system controller 20 and power source 22 are illustrated outside/external to the patient body 12, the driveline 26, the system controller 20 and/or the power source 22 can be partially or fully implantable within the patient 12, as separate components or integrated with the VAD 14. Examples of such modifications are further described in U.S. Pat. Nos. 8,562,508 and 9,079,043, all of which are incorporated herein by reference for all purposes in their entirety.

With reference to FIG. 3, the VAD 14 has a circular shaped housing 110 and is shown implanted within the patient 12 with a first face 111 of the housing 110 positioned against the patient's heart 30 and a second face 113 of the housing 110 facing away from the heart 30. The first face 111 of the housing 110 includes an inlet cannula 112 extending into the left ventricle LV of the heart 30. The second face 113 of the housing 110 has a chamfered edge 114 to avoid irritating other tissue that may come into contact with the VAD 14, such as the patient's diaphragm. To construct the illustrated shape of the puck-shaped housing 110 in a compact form, a stator 120 and electronics 130 of the VAD 14 are positioned on the inflow side of the housing toward first face 111, and a rotor 140 of the VAD 14 is positioned along the second face 113. 10 The stator 120 can be controlled as discussed in U.S. Pat. No. 6,351,048, the entire contents of which are incorporated herein by reference for all purposes. The control electronics 130 and the stator 120 receive electrical power from a remote power supply via a cable 119. This positioning of the stator 120, electronics 130, and rotor 140 permits the edge 114 to be chamfered along the contour of the rotor 140, as illustrated in at least FIG. 3, for example. Further related patents, namely U.S. Pat. Nos. 5,708,346, 6,053,705, 6,100,618, 6,222,290, 6,249,067, 6,278,251, 6,351,048, 6,355,998, 6,634,224, 6,879,074, and 7,112,903, all of which are incorporated herein by reference for all purposes in their entirety.

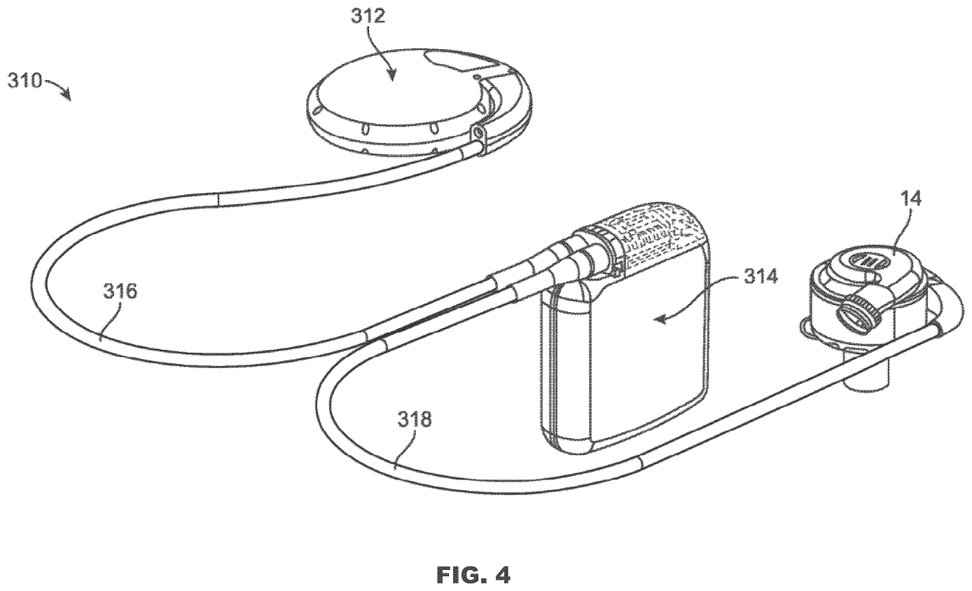

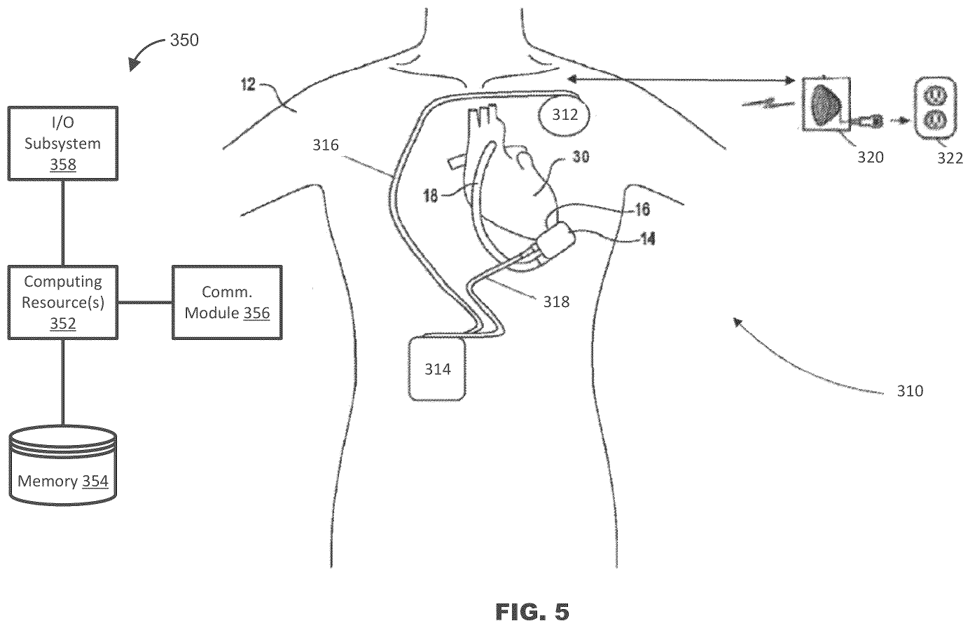

FIG. 4 shows a mechanical circulatory support system 310 that includes implantable transcutaneous energy transmission system (TETS) receiver 312, an implantable controller 314, the VAD 14, a first electrical cable 316, and a second electrical cable 318, in accordance with many embodiments. The implantable controller 314 is configured similar to the external controller 20, but is further configured to be implanted. The TETS receiver 312 can be, for example, a receiver, a resonator, and inductive coil or the like, for receiving transmitted electrical power used to power the circulatory support system 310. In some embodiments, the resonant frequency of the TETS receiver 312 is in a range of 100 kHz to 10 MHz.

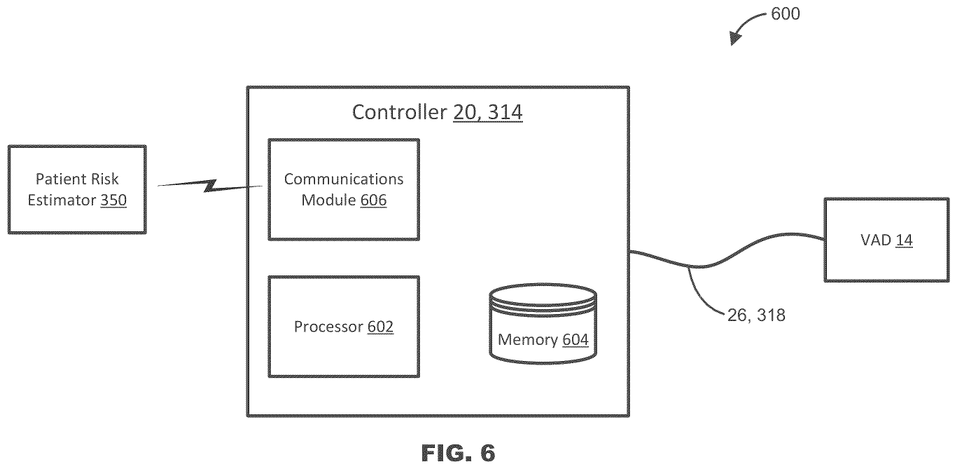

FIG. 5 illustrates the mechanical circulatory support system 310 using the implantable controller 314 in conjunction with a ventricular assist device (VAD) 14 implanted in a patient 12. In many embodiments described herein, the mechanical circulatory support system 310 includes the implantable VAD 14 coupled to a patient's heart 30, the implantable controller 314 that controls operation of the VAD 14 and powers the VAD 14, and an implantable transcutaneous energy transfer system (TETS) power receiver 312 that supplies power to the implantable controller 314. The VAD 14 as illustrated can be a centrifugal flow blood pump, or an axial flow blood pump, which can be operated in a pulsatile mode that produces a blood pressure pulse via an increase in the rotational speed of the VAD or in a constant speed mode in which the VAD rotational rate is held constant.

Referring to FIG. 4 and FIG. 5, the mechanical circulatory support system 310 further includes the implantable ventricular cuff 16, the implantable outflow cannula 18, an external TETS power transmitter 320, an implantable controller-to-VAD connection cable 318, and an implantable TETS receiver-to-controller connection cable 316. The VAD 14 can be coupled to an apex of the left ventricle via the ventricular cuff 16, as illustrated, or the right ventricle, or a separate VAD can be attached to each of the ventricles of the heart 30. Accordingly, depending on the medical application, the mechanical circulatory support system 310 can also be referred as a fully implantable left ventricular assist system (FILVAS) 310. Examples of such FILVAS systems are further described in U.S. Pat. Nos. 8,562,508 and 9,079,043, all of which are incorporated herein by reference for all purposes in their entirety.

The VAD 14, as described in more detail below, can be capable of pumping the entire flow of blood delivered to the left ventricle from the pulmonary circulation (i.e., up to 10 liters per minute). The VAD 14 can be attached to the heart 30 via the ventricular cuff 16, which can be sewn to the heart 30 and coupled to the VAD 14. In the illustrated embodiment, the output of the VAD 14 connects to the ascending aorta via the outflow cannula 18 so that the VAD 14 effectively diverts blood from the left ventricle and propels it to the aorta for circulation through the rest of the patient's vascular system.

The controller-to-VAD connection cable 318 connects the VAD 14 to the implantable controller 314, which monitors the system 310 operation The implantable controller 314 is configured to supply power to and control operation of the VAD 14. The implantable controller 314 is configured to be implanted within the patient 12 in a suitable location spaced apart from the VAD 14 and operatively coupled with the VAD 14 via the controller-to-VAD connection cable 318.

The TETS power receiver 312 is configured to wirelessly receive power transmitted by the external TETS power transmitter 320, which is outside the body for powering operation of the system 310. The TETS power receiver 312 is configured to be implanted within the patient 12 in a suitable location spaced apart from the VAD 14 and the controller 314. The TETS power receiver 312 is operatively coupled with and supplies power to the controller 314 via the implantable TETS power receiver-to-controller connection cable 316. Further, the TETS power transmitter 320 is configured to be coupled to an electric power source 322 such as an electrical wall outlet or other suitable external power sources.

### Patient Risk Estimation System

A patient risk estimator 350, also referred to as the patient risk estimation system 350 can interact with the mechanical circulatory support system 310 of FIG. 4 and FIG. 5, or with the mechanical circulatory support system 10 of FIG. 1. The patient risk estimator 350 can be configured to receive inputs from one or several users and/or from one or several devices and/or systems such as, for example, the mechanical circulatory support system 10, 310, and with these inputs, generate risk predictions for an individual patient. These risk predictions can be generated with a machine learning model, and specifically with a multistate model. These risk predictions can be generated, in some embodiments, on a daily basis.

The patient risk estimator 350 can include one or several computing resources 352. The computing resources can comprise one or several servers, computers, computing devices, processors, or the like. The processor can include a microprocessor, such as one or several Central Processing Units (CPUs) and/or one or several Graphics Processing Units (GPUs). The processor can be a commercially available microprocessor from Intel®, Advanced Micro Devices, Inc.®, Nvidia Corporation®, or the like.

The one or several computing resources 352 can be configured to receive data from one or several sources, some or all of which data can be used as features for the generation of the risk prediction, ingest the features into a machine learning model, make a risk prediction with the machine learning model, and output the risk prediction. In some embodiments, the one or several computing resources can be configured to generate one or several control signal to affect operation of an implanted device, such as an implanted VAD, based on the risk prediction. These one or several control signals can be provided by the one or several computing resources 352 to the controller 20, 314 of the mechanical circulatory support system 10, 310 to affect operation of the VAD 14. In some embodiments, the computing resource 352 can be controlled according to computer code and/or instructions that can be stored in memory accessible by the computing resource 352.

The patient risk estimator 350 can include memory 354 communicatingly coupled to the computing resource 352. The memory 354 can include primary and/or secondary memory. The memory 354 can include, for example, cache memory, RAM, ROM, PROM, EPROM, EEPROM, one or several solid-state drives (SSD), one or several hard drives or hard disk drives, or the like. Thus, in some embodiments, the memory 354 can include volatile and/or non-volatile memory.

The memory 354 can include instructions in the form of computer code that can be accessible by the computing resource 352, and that when executed by the computing resource 352, cause the computing resource to generate the risk prediction. The memory 354 can further include a machine learning model that is accessible by the computing resource 354. The machine learning model can be trained to, upon receiving features, output a risk prediction of a patient experiencing one or adverse outcomes associated with one or several states of the machine learning model. The machine learning model can comprise a multistate model, and specifically can comprise a logistic regression multistate model. The multistate model can include a plurality of states. In some embodiments, some or all of the plurality of states can correspond to a patient condition, an adverse event or outcome, or the like.

The patient risk estimator 350 can include a communications module 356. The communications module 356 can be configured to communicate with one or several devices and/or systems, including one or several implanted devices. In some embodiments, the communications module 356 can be configured to communicate with the controller 20, 314 of the VAD 14 to receive information relating to one or several current operating parameters of the VAD 14 and/or to provide one or several control signals to the VAD 14 to affect one or several operating parameters of the VAD 14. In some embodiments, these one or several operating parameters, which can be used as features for generation of a risk prediction, can include, for example, a pump pulsatility index; a pump power; a pump flow (l/m), and a pump speed. In some embodiments, the pump pulsatility index can indicate a degree to which the pump volume pulses (increased and decreases) with time. This pulsing of the pump volume can be synchronous with the pumping of the heart and/or can be asynchronous with the pumping of the heart. The pump power can characterize the power consumed by the pump, and the pump speed can character the speed at which the pump is operating.

The communications module 256 can be configured to wired or wirelessly communication with the controller 20, 314. In some embodiments, the communications module 356 can comprise a transmitter, a receiver, a transceiver, and/or one or several antennas.

The patient risk estimator 350 can comprise an Input/Output Subsystem (“I/O subsystem”) 358. The I/O subsystem 358 can comprise one or several features, which can be one or several hardware features, configured to provide outputs to a user and to receive inputs from the user. The I/O subsystem 358 can include, for example, one or several monitors, displays, screens, touchscreens, keyboards, keypads, mouses, speakers, microphones, cameras, or the like.

The I/O subsystem 358 can be controlled by to provide a user interface (“UX”), whereby the user can interact with and control the operation of the computing resource. In some embodiments, the UX can be controlled to output the risk prediction and/or one or several trendlines showing the risk predictions over time to the user.

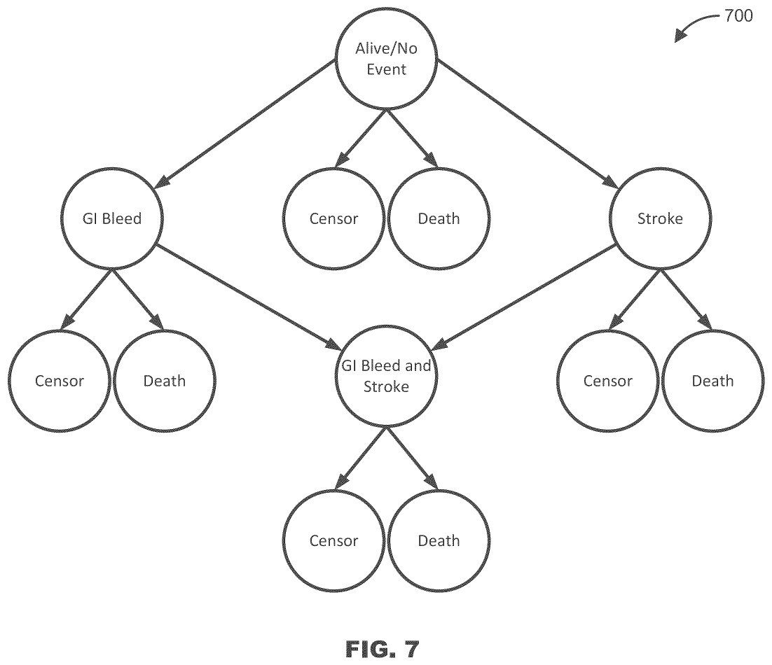

Referring to FIG. 6, a schematic depiction 600 of one embodiment of the controller 20, 314 of the mechanical circulatory support system 10, 310 is shown. The controller 20, 314 is communicating coupled to the VAD 14 via the driveline 26 and/or the controller-to-VAD connection cable 318. The controller 20, 314 can be configured to receive information from the VAD 14 and to generate one or several control signals to affect aspects of operation of the VAD 14. The controller 20, 314 includes a processor 602. The processor 602 can include a microprocessor, such as one or several Central Processing Units (CPUs) and/or one or several Graphics Processing Units (GPUs). The processor 602 can be a commercially available microprocessor from Intel®, Advanced Micro Devices, Inc.®, Nvidia Corporation®, or the like.

The processor 602 can be receive information from the VAD 14 and to generate one or several control signals to affect one or several operating parameters of the VAD 14. The processor 602 can be controlled according to computer code and/or instructions that can be stored in memory 604 accessible by the processor 602.

The controller 20, 314 can include memory 604 communicatingly coupled to the processor 602. The memory 604 can include primary and/or secondary memory. The memory 604 can include, for example, cache memory, RAM, ROM, PROM, EPROM, EEPROM, one or several solid-state drives (SSD), one or several hard drives or hard disk drives, or the like. Thus, in some embodiments, the memory 604 can include volatile and/or non-volatile memory.

The memory 604 can include instructions in the form of computer code that can be accessible by the processor 602, and that when executed by the processor 602, cause the processor 602 to take one or several actions.

The controller 20, 314 can include a communications module 606. The communications module 606 can be configured to communicate with, for example, the patient risk estimator 350. In some embodiments, the communications module 606 can be configured to provide outputs indicating one or several current operating parameters of the VAD to the patient risk estimator 350 and receive one or several control signals from the patient risk estimator 350 affecting one or several operating parameters of the VAD 14. In some embodiments, these one or several operating parameters can include, for example, the pump pulsatility index; the pump power; and the pump speed.

The communications module 606 can be configured to wired or wirelessly communication with the patient risk estimator 350. In some embodiments, the communications module 606 can comprise a transmitter, a receiver, a transceiver, and/or one or several antennas.

Referring to FIG. 7, a schematic depiction 700 of one embodiment of the multistate model is shown. Specifically, FIG. 7 depicts the hierarchy of some of the states used by the multistate model to track the progression of a patient. The model shown in FIG. 7 is simplified and does not model a recurrent event. However, in some embodiments, the multistate model can be configured to capture and/or model recurrent events.

As seen, in FIG. 7, the states include a state for alive and with no adverse event, a state for a gastrointestinal bleed (“GI Bleed”), a state for death of the patient, a state for a stroke, a state for both a GI Bleed and stroke, and a state for censor. In some embodiments, the censor state can be state indicating a patient that has exited from receipt of further treatment and/or monitoring for the VAD 14. For example, a patient that has had the VAD 14 removed can be identified as in the censored state. For example, a patient who receives the VAD 14 as a temporary treatment until they are able to receive a heart transplant can be identified as in the censored state when they have the VAD 14 removed as part of receiving a heart transplant. Similarly, a patient who reaches the end of a tracking period, and in some embodiments, who reaches the end of the tracking period without an adverse event can be identified as in the censored state. The tracking period can, in some embodiments, last for one year, two years, three years, four years, five years, ten years, twenty years, or any other or intermediate number of years. During this period, the patient can be monitored, and specifically can be monitored for one or several adverse events. Thus, in some embodiments, a patient reaching the end of the tracking period, and specifically reaching the end of the tracking period without an adverse event, can be identified as in the censored state. Lastly, a patient can be identified as in the censored state if the patient ceases and/or terminates support for the VAD 14.

In the multistate model, patient disease progression can be modeled as different categorical states depending on the number of various adverse events the patient suffered from. In some embodiments, patients start in “Alive without Event” state. Once the first adverse event of interest occurred, a death event happened, or the patient is censored, the patient is moved from “Alive without Event” to the next state.

In the multistate model, the patient can have more than one incident of the same type of adverse event. For example, a patient that has more than 1 incidents of the GI Bleeding event remains in the GI Bleeding state before transitioning to the next state.

In some embodiments of the multistate model, only adverse events of interest (i.e., GI Bleeding and Stroke) are tracked. These two types of events can be tracked in the multistate model as the can, in some embodiments, occur relatively frequently amongst VAD patients and as these two types of events can be clinically manageable using medications or known treatment. In some embodiments, these states are used to model all the patients throughout the two years of follow-up as studied in this analysis.

Although the depiction of the multistate model in FIG. 7 only depicts five unique states, in some embodiments, the multistate model can track a first number of states, and can output a risk prediction relevant to a second number of states. In some embodiments, the first number of states can be greater than the second number of states, or in other words, the multistate model can track a greater number of states than the number of states for which a risk prediction is outputted. In some embodiments, the tracking of a greater number of states than the number for which a risk prediction is outputted can improve the performance of the multistate model.

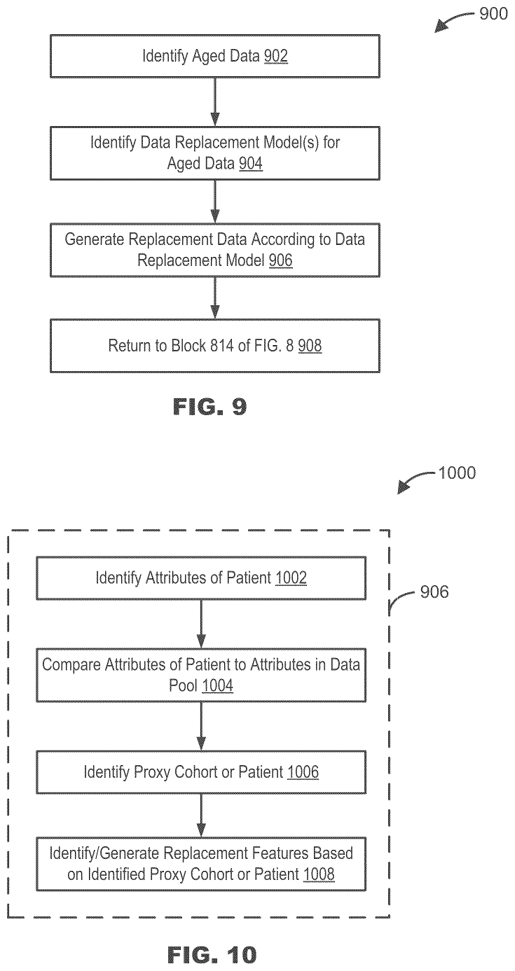

Referring now to FIG. 8, a flowchart illustrating one embodiment of a process 800 for post-operative risk mitigation in patients with an implanted Ventricular Assist Device (VAD) is shown. The process 800 can be performed by all or portions of the patient risk estimator 350.

The process 800 begins at block 802, wherein it is determined to generate a prediction for a patient of a likelihood of that patient developing a condition, or in other words, wherein it is determined to generate a risk prediction of that patient experiencing an adverse event that is associated with a state in machine learning, multistate model. In some embodiments, this determination can be made by the computing resource 352 due to the computing resource 352 detecting and/or identifying the occurrence of a triggering event. In some embodiments, this triggering event can be the passage of a predetermined amount of time, the receipt of a piece of data relevant to the patient, or the like. In some embodiments, for example, the patient risk estimator 350 can be configured to generate a risk prediction at a regular interval such as, for example, every other week, once a week, every sixth day, every fifth day, every fourth day, every third day, every other day, every day, twice a day, four times a day, eight times a day, twelve time a day, between 2 and 1500 times per month, or at any other or intermediate interval. In some embodiments, the patient risk monitor 350 can be configured to generate a risk prediction when a triggering event occurs. This event can include, for example, receipt of data indicative of a change in a patient state and/or indicative of the patient experiencing an adverse event. In some embodiments, this triggering event can be, for example, receipt of certain updated relating to the patient and relevant to the risk prediction. In some embodiments, the determination to generate a risk prediction can occur at regular fixed intervals, unless there is an intervening triggering event leading to generation of an intermediate risk prediction between regularly scheduled risk predictions.

At step 804, features associated with the patient who received the VAD implant are received. Each of the received features can, in some embodiments, relate to an attribute associated with the patient that received the implanted VAD. In some embodiments, the plurality of parameters can include one or more of: a baseline feature indicating a pre-operative condition of the patient; an implantation feature indicating a condition of the patient during implantation of the VAD; and a post-implantation feature indicative of a post-operative condition of the patient. In some embodiments, at least one of the post-implantation features is time-varying. In some embodiments, the post-implantation feature can identify an operating attribute of the VAD, which operating attribute can include, for example, a pump pulsatility index; a pump power; and a pump speed.

In some embodiments, these features can be received via user input by the patient risk estimator 350 via the I/O subsystem 358 and/or can be received from one or several systems and/or devices with which the patient risk estimator 350 is communicatingly connected. In some embodiments, for example, the patient risk estimator 350 is communicatingly connected with the VAD, and one or several operating features of the VAD implanted in the patient can be received by the patient risk estimator 350. In some embodiments, these features can be received before or after the determination of block 802.

At block 806, the received features are evaluated to determine if they are aged. In some embodiments, this evaluation can be performed by the computing resource 352 of the patient risk estimator 350. An aged feature is time variable data that is older than a predetermined amount of time and/or that has been previously used in generation of a risk prediction. Thus, in some embodiments, the computing resource 352 can determine if a feature is aged by comparing a date associated with the feature, such as, for example, the date that a measurement indicated by the feature was taken, the date that a feature was created, the date a treatment was provided or a medication was administered, and/or the date that a feature was received with a threshold delineating between features that are aged and features that are not aged. When the amount of time since the date of a feature is greater than the threshold, then that feature can be identified as aged.

Other criteria can be used in determining whether a feature is aged, including, whether the feature has been previously used in a risk prediction. In such an embodiment, the computing resource can determine whether the feature was previously used in a risk prediction, and if so, can identify that feature as aged.

At decision block 808, it is determined if one or several of the features associated with the patient and that will be used in the generation of the risk prediction are aged. In some embodiments, this can include determining if one or several of the features associated with the patient and that will be used in the generation of the risk prediction are aged and invalid. Data is aged and invalid when the data is aged, and when new data corresponding to the aged data is available. Thus, in some embodiments, data that is aged can be valid if it has not yet been replaced by new data.

This determination of whether one or several of the features associated with the patient

and that will be used in the generation of the risk prediction are aged can be made by the patient risk estimator 350, and specifically by the computing resource 352. In some embodiments, this determination can be made based on the outcome of the evaluation performed in block 806. If it determined that one or several of the features to be used in the generation of the risk prediction is aged, then the process 800 proceeds to block 810 and continues at block 902 of FIG. 9.

Returning to decision block 808, if it is determined that none of the received features are aged, and/or that none of the received features are aged and invalid, then the process 800 proceeds to block 814, wherein a prediction of a likelihood of a patient developing a condition associated with a state is generated. In some embodiments, this can include generating a prediction of a risk of a patient experiencing an adverse event that is associated with one of the states of the machine learning model. In some embodiments, the risk prediction can indicate a likelihood of the patient developing at least one of the conditions corresponding to the plurality of states in a predetermined time period. This predetermined time period can include, for example, one or several days, weeks, months, years, or the like. In some embodiments, this predetermined time period can be within the next month, the next three months, the next year, the next two years, or any other or intermediate time period.

This risk prediction can be generated by the patient risk estimator 350, and specifically by the computing resource 352 of the patient risk estimator. In some embodiments, the generation of the risk prediction can include inputting some or all of the features, including non-aged features, aged features, and/or replacement features into the machine learning model, and the outputting of the risk prediction from the machine learning model. In some embodiments, these inputs can include a time-frame related input. The time-frame related input can identify the amount of time post-implant. In some embodiments, this amount of time can be placed in one of a plurality of tranches including, for example, short-term post implant, mid-term post implant, and long-term post implant.

In some embodiments, the risk prediction can be a daily risk prediction, in other words, the risk prediction can be generated at a once-a-day interval. In some embodiments, and as previously mentioned, the multistate model can track a first number of states and/or generate a risk prediction for a first number of states, and can output a risk prediction relevant to a second number of states. In some embodiments, the first number of states can be greater than the second number of states, or in other words, the multistate model can track a greater number of states, and can generate risk prediction for the patient achieving a greater number of states than the number of states for which a risk prediction is outputted. Thus, in some embodiments, generating the risk prediction can include generating with the multistate model the daily prediction of the likelihood of the patient developing a first set of conditions corresponding to a first set of states.

At step 816, an indicator of the generated likelihood of the patient developing at least one condition associated with at least one state is output via the user interface. In some embodiments, the computing resource 352 can control the user I/O subsystem 358, and specifically can control the UX on the I/O subsystem 358 to display the risk prediction generated in block 814. In some embodiments, this can include displaying a risk prediction comprising a plurality of predictions, one prediction for each of a plurality of adverse events. In some embodiments, the UX can be controlled to output an indicator of the likelihood of the patient developing the at least one of the conditions in the predetermined time period. In some embodiments, controlling the user interface to output the indicator of the likelihood of the patient developing the at least one of the conditions in the predetermined time period can include controlling the user interface to output the indicator of the likelihood of the patient developing a second set of conditions corresponding to a second set of states in the predetermined time period. In some embodiments, the first set of conditions corresponding to the first set of states for which a risk prediction is generated is greater than the second set of conditions corresponding to the second set of states that are output via the indicator of the UX.

At step 818, a control signal is generated and sent to the implanted device, which control signal affects operation of the implanted device. In some embodiments, the implanted device can comprise the VAD 14, and the control signal can be a control signal configured to affect an operating attribute of the VAD 14 such as, for example, the pump pulsatility index; the pump power; and the pump speed. In some embodiments, the control signal can be generated by the computing resource 352 and can be sent to the implanted device, and specifically can be sent to the VAD 14 via the communications module 356 of the patient risk estimator 350. The control signal can be received by the communications module 606 of the controller 20, 314. Based on this received signal, the controller 20, 314 can change an operating parameter of the VAD 14 according to the received control signal.

In some embodiments in which the generation and display of a trendline is desired, the process 800 can proceed to decision block 820, alternatively, if generation and display of a trendline is not desired, the process 800 can proceed directly to block 828, which will be discussed below. At decision block 820, it is determined if there are multiple risk predictions that have been generated for the patient. If it is determined that multiple risk predictions have not been generated, then the process 800 proceeds to block 828 and proceeds as discussed below. If multiple risk predictions have been generated for the patient, then the process 800 proceeds to block 822, wherein at least one trendline is generated based on the multiple risk predictions, and the at least one trendline is output via the UX. In some embodiments, this can include controlling the UX to output at least one trendline based on the aggregate of generated daily predictions. In some embodiments, this at least one trendline can comprise a single trendline, and in some embodiments, the at least one trendline can comprise a plurality of trendlines, each of which trendlines is associated with one of the at least one conditions corresponding to one of the plurality of states.

Each of the at least one trendlines indicates time-varying risk predictions. Thus, one trendline indicates a change in risk with respect to the associated at least one condition corresponding to the one of the plurality of states. Thus, the trendline can indicate an increase of risk over time or a decrease in risk over time. In some embodiments, the patient risk estimator 350 can evaluate the trendline to determine whether risk is increasing or decreasing, and can take action based on this determination. In some embodiments, this can include determining whether the trendline is maintaining a consistent risk prediction, is sloping towards increased risk, is sloping towards decreased risk, or is changing. In some embodiments, for example, the patient risk estimator 350 can evaluate the trendline for indication of a change in slope, and particularly for a change in slope towards an increased risk prediction.

At decision block 824, it is determined whether to alert or modify treatment due to instantaneous risk, due to the slope of the trendline, and/or due to a change in the slope of the trendline. In some embodiments, this determination can include comparing one or several attributes of the risk and/or of the trendline to one or several thresholds. This can include, for example, comparing the current risk prediction to one or several threshold delineating between acceptable risk levels and unacceptable risk levels, comparing the slope of the trendline to one or several thresholds delineating between acceptable slopes and unacceptable slopes of trendlines, and/or comparing a change in the slope of the trendline to one or several thresholds delineating between acceptable changes in trendline slope and unacceptable changes in trendline slope. In some embodiments, the trendline slope and/or the change in the trendline slope can indicate an increased risk of occurrence an adverse event or a decreased risk of occurrence of an adverse event.

If it is determined not to alert or modify treatment, then the process 800 proceeds to block 828 and proceeds as discussed below. If it is determined to alert or modify treatment due to the risk prediction, due to the slope of the trendline, and/or due to a change in the slope of the trendline, then the process 800 proceeds to block 826, wherein an alert is generated and/or treatment is modified based on this determination.

In some embodiments, the alert can be in the form of an indicator in the UX which can indicate the excessive risk, the slope of the trendline, and/or the change in the slope of the trendline. In some embodiments, for example the computing resource 352 can control the I/O subsystem 358 to generate and display an indicator providing the alert to the user.

In some embodiments, a treatment of the patient can be modified to compensate for and/or to address the risk of the adverse event and/or the underlying causes of that risk of the occurrence of the adverse event. In some embodiments, this can include identifying one or several parameters giving rise to the elevated risk, the unacceptable trendline slope, and/or the unacceptable change in the trendline slope, and generating a treatment to affect these parameters. For example, if it is determined that the patient risk is due to a patient blood pressure, then a treatment may be generated to change the patient blood pressure to decrease the risk of occurrence of an adverse event. In some embodiments, this can include modifying operation of the implanted device, providing one or several medications, providing a treatment, or the like. In some embodiments, modifying of the treatment of the patient can include the automatic identification of one or several treatments and/or medications to address the source of the elevated risk, automatically scheduling the one or several treatments, and/or automatically generating one or several prescriptions or proposed prescriptions.

At block 828, the occurrence of another trigger for risk prediction generation is determined. In some embodiments, this can include determining that it is time for generation of a new risk prediction and/or that an intervening event has occurred thereby triggering generation of a new risk prediction. This determination can be made by the computing resource. After the determination of the occurrence of the trigger event, the process returns to block 804, and proceeds as outlined above.

Due to the repeated generation of risk predictions, in some cases daily generation of risk predictions, a plurality of risk predictions can be generated at regular intervals over a period of time. For example, in some embodiments, a risk prediction may be generated each day for a plurality of days.

Referring to FIG. 9, a flowchart illustrating one embodiment of a process 900 for generating replacement features is shown. The process 900 can be performed as a part of process 800, and specifically can be performed subsequent to the process 800 moving to block 810. The process 900 begins at block 902, wherein aged data and/or aged features are identified. In some embodiments, this can include identifying time variable data that is older than a predetermined amount of time and/or that has been previously used in generation of a risk prediction. In some embodiments, this can include the computer resource 352 evaluating the received data to identify which of the received data is aged.

At block 904, one or several data replacement models are identified. In some embodiments, a data replacement model can be a vehicle through which replacement data can be generated, created, and/or received. This replacement data can then, in some embodiments, be used in the place of the aged data. In some embodiments, different types of aged data can have different data replacement models. In some embodiments, aged data relating to an attribute of a patient, can use a replacement model based on a proxy cohort of one or several patients sharing one or several attributes with the patient for whom the replacement data is being generated. In some embodiments, the data replacement model can be a model that selects a most recent value for use as a replacement model, and in some embodiments, the data replacement model can be configured to determine to generate new data in the place of at least one of the aged features, and schedule an action to generate that new data.

In some embodiments, the computing resource 352 can identify the data replacement model according to one or several lookup tables linking one or several attributes to one or several replacement models, and/or via one or several rules stored in a rule database in the memory 354. In some embodiments, for example, the computing resource 352 can select one of the aged features, and query the lookup table for the data replacement model for that aged feature. Alternatively, the computing resource 352 can select one of the aged features, can identify and retrieve one or several rules applicable to that aged feature, and can determine the data replacement model for that aged feature based on the one or several rules applicable to that aged feature.

At block 906, replacement data is generated according to the identified data replacement model(s). Thus, for embodiment in which there is a single aged feature, or in which all of the aged features share a common data replacement model, that single data replacement model is used to generate replacement data. Alternatively, if there is more than one aged feature and the aged features do not share a common data replacement model, then the replacement data can be generated with the plurality of replacement models.

At block 908, the process 900 proceeds to block 814 of FIG. 8, and continues as outlined above. In some embodiments, this can include the generation of features with the replacement data generated by process 900.

With reference now to FIG. 10, a flowchart illustrating one embodiment of a process 1000 for generating replacement features based on a proxy cohort or proxy patient is shown. The process 1000 can be performed as a part of, or in the place of the step of block 906 of FIG. 9. The process 1000 can be performed by the patient risk estimator 350 and/or by the computing resource 352.

The process 1000 begins at block 1002, wherein attributes of the patient having the aged data are identified. These attributes can include, for example, patient sex, age, weight, blood pressure, heart rate, medical history, or the like. These attributes can, in some embodiments, be identified by the computing resource 352 from, for example, memory 354.

At block 1004, the attributes of the patient identified in block 1002 are compared to attributes in the data pool. In some embodiments, the data pool can comprise attributes of a plurality of patients that have previously received an implanted VAD. This comparison of the attributes of the patient to attributes in the data pool can facilitate in identifying a proxy cohort and/or a proxy patient.

At block 1006, a proxy cohort of patients and/or a proxy patient is identified. In some embodiments, the proxy cohort of patients, also referred to herein as a patient cohort, can be selected based on a commonality of at least one attribute with the patient having the aged data and/or having received the implanted VAD 14. In some embodiments, a proxy cohort of patients can be a group of patients having attributes stored in the data pool and having sufficient similarity to the patient with the aged data as to allow them to be used as a source for replacement data for the patient's aged data. Likewise, a proxy patient is a patient having attributes stored in the data pool and identified as sufficiently similar to the patient with the aged data as to allow the proxy patient to serve as a source for replacement data for the patient's aged data.

At block 1008, replacement data is identified and/or generated based on data and/or attributes of the identified proxy cohort and/or of the proxy patient. In some embodiments, for example, the replacement data can be the mean, median, or mode of data from the proxy cohort and corresponding to the aged data. In some embodiments, for example, the replacement data can be the data from the proxy patient corresponding to the aged data.

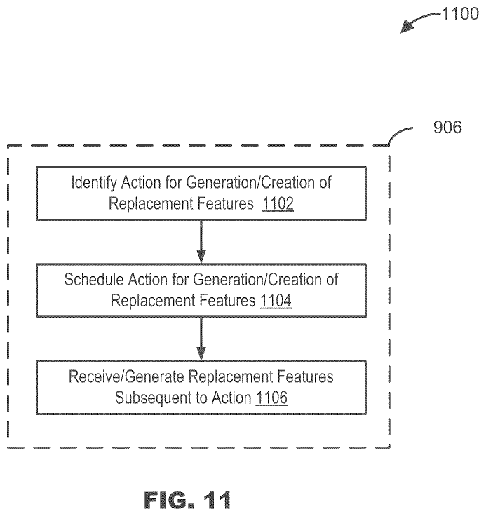

With reference to FIG. 11, a flowchart illustrating one embodiment of a process 1100 for generating replacement features via scheduling of an action is shown. The process 1100 can be performed as a part of, or in the place of step 906 of FIG. 9. The process 1100 can be performed by the patient risk estimator 350 and/or the computing resource 352.

At block 1102, an action for generation and/or creation of replacement data is identified. In some embodiments, this action can include, for example, a physician visit to collect new patient data, a patient treatment, or the like. In some embodiments, the action can be identified based on attributes of the aged data, and in some embodiments, can be identified via a lookup table stored in, for example, the memory 354.

At block 1104, the action for generation and/or creation of the replacement data is scheduled. In some embodiments, this can include scheduling a time for this action and notifying participants of the scheduled time.

At block 1106, the replacement data can be received by the patient risk estimator 350 subsequent to the action of block 1104. In some embodiments, after the new data has been received subsequent to the scheduled actin, the generation of the risk prediction can proceed. Thus, in such an embodiment, the generation of the risk prediction can be delayed until the new data is received, or alternatively, the generation of the risk prediction can proceed with the aged data.

### Exemplary Embodiment

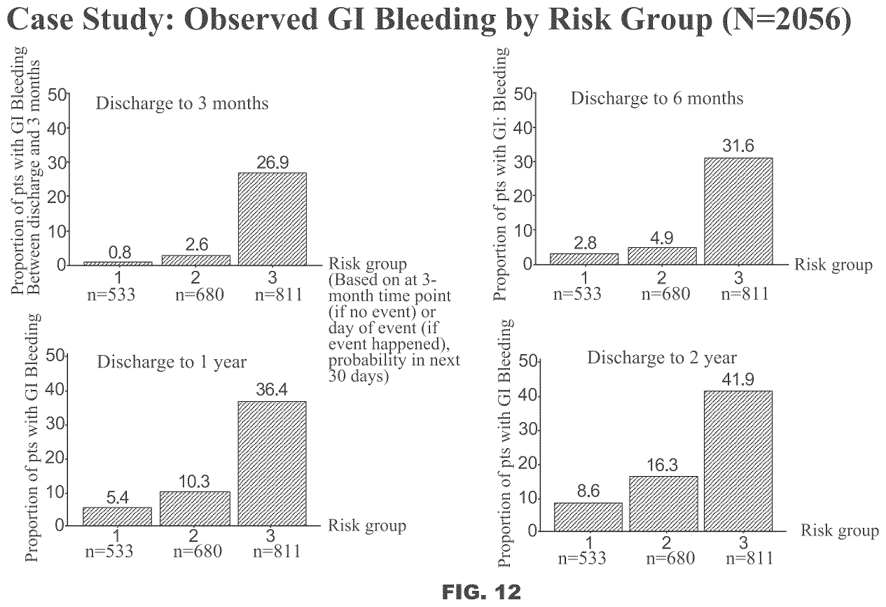

Details of an exemplary embodiment, and results achieved with that embodiment are described below. With reference now to FIG. 12 to FIG. 15, results of a case study of 2056 patients who received an implanted VAD are shown. Each of FIG. 12 to FIG. 14 shows four charts, each of which indicates the frequency of a complication for three different risk groups within a time period.

FIG. 12 depicts Observed GI Bleeding by Risk Group, and includes a first chart depicting the proportion of patients with GI bleeding between discharge to three months, a second chart depicting the proportion of patients with GI bleeding between discharge to six months, a third chart depicting the proportion of patients with GI bleeding between discharge to one year, and a fourth chart depicting the proportion of patients with GI bleeding between discharge to two years. Each of these charts shows patients divided into one of three risk groups, a low-risk group including 533 patients, a medium risk group including 680 patients, and a high-risk group including 811 patients.

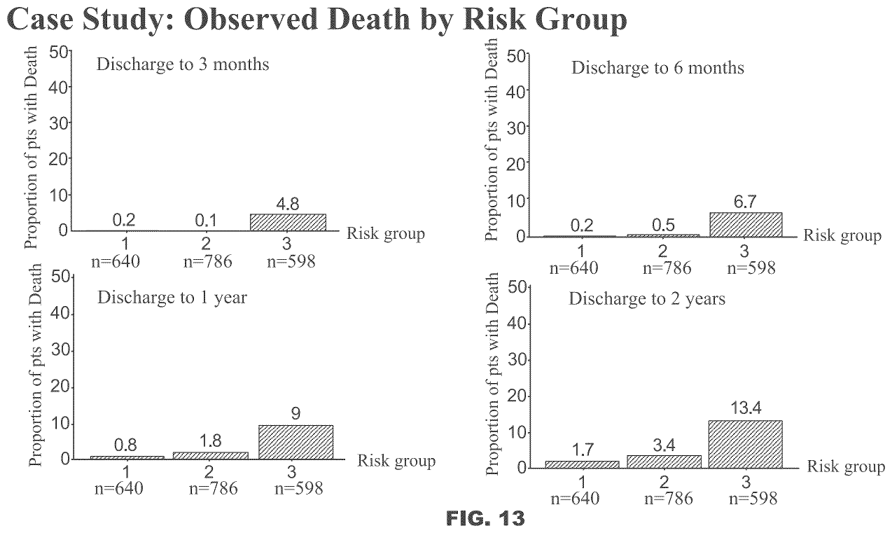

FIG. 13 depicts Observed Death by Risk Group, and includes a first chart depicting the proportion of patients dying between discharge to three months, a second chart depicting the proportion of patients dying between discharge to six months, a third chart depicting the proportion of patients dying between discharge to one year, and a fourth chart depicting the proportion of patients dying between discharge to two years. Each of these charts shows patients divided into one of three risk groups, a low-risk group including 640 patients, a medium risk group including 786 patients, and a high-risk group including 598 patients.

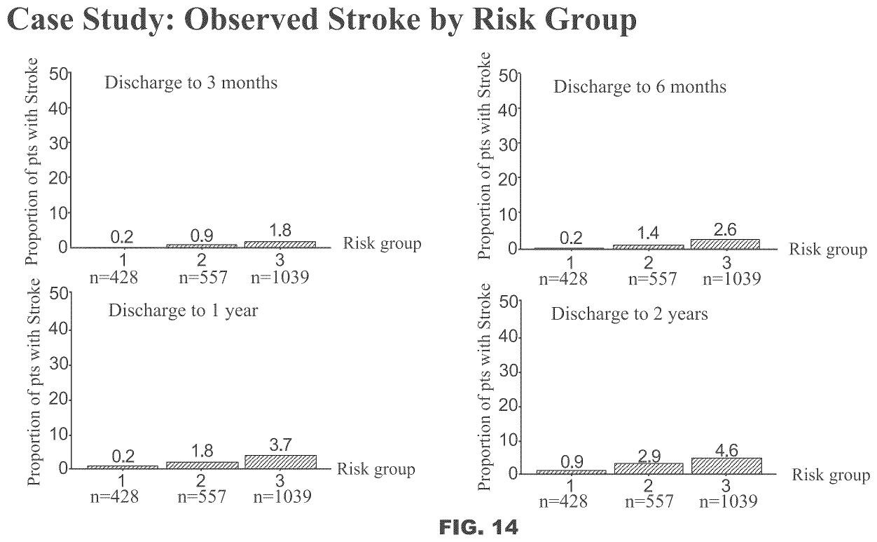

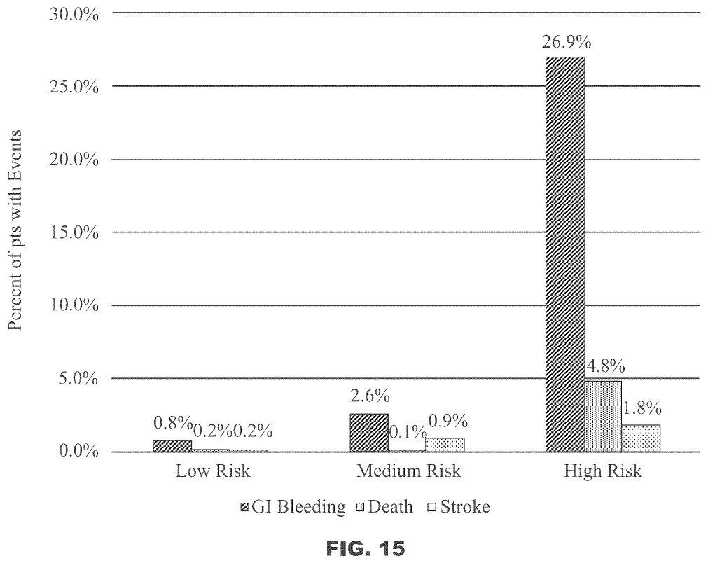

FIG. 14 depicts Observed Stroke by Risk Group, and includes a first chart depicting the proportion of patients having a stroke between discharge to three months, a second chart depicting the proportion of patients having a stroke between discharge to six months, a third chart depicting the proportion of patients having a stroke between discharge to one year, and a fourth chart depicting the proportion of patients having a stroke between discharge to two years. Each of these charts shows patients divided into one of three risk groups, a low-risk group including 428 patients, a medium risk group including 557 patients, and a high-risk group including 1039 patients. FIG. 15 depicts Observed Adverse Events by Risk Group. This chart includes the results of the first chart in each of FIG. 12, FIG. 13, and FIG. 14 combined together by risk group. As seen in each of these figures, adverse events increase with passing time, and the GI intestinal bleeding is the highest frequency adverse event.

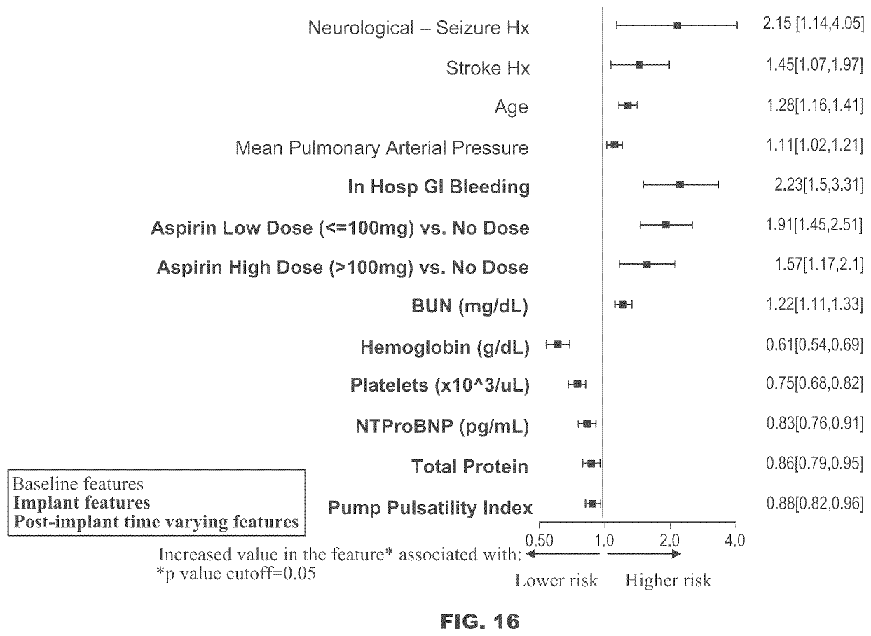

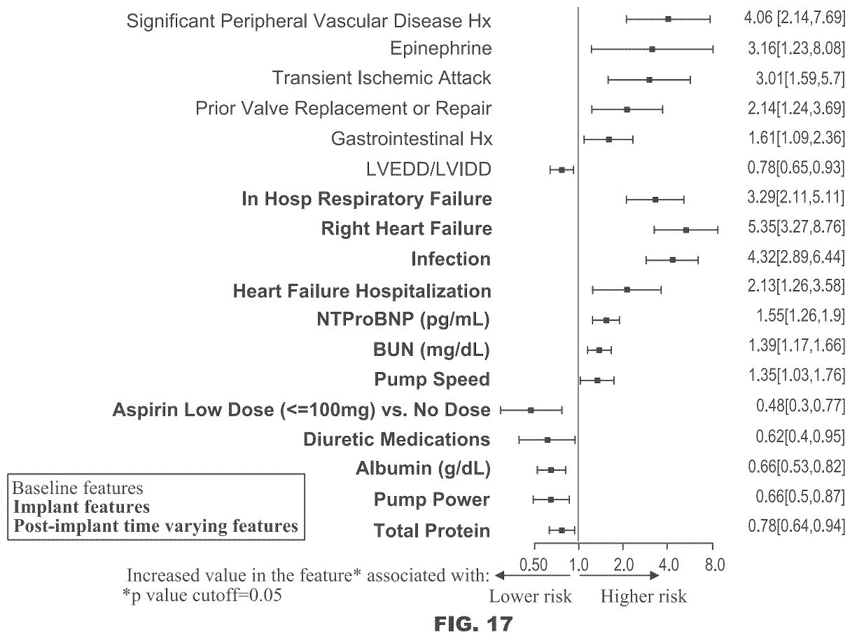

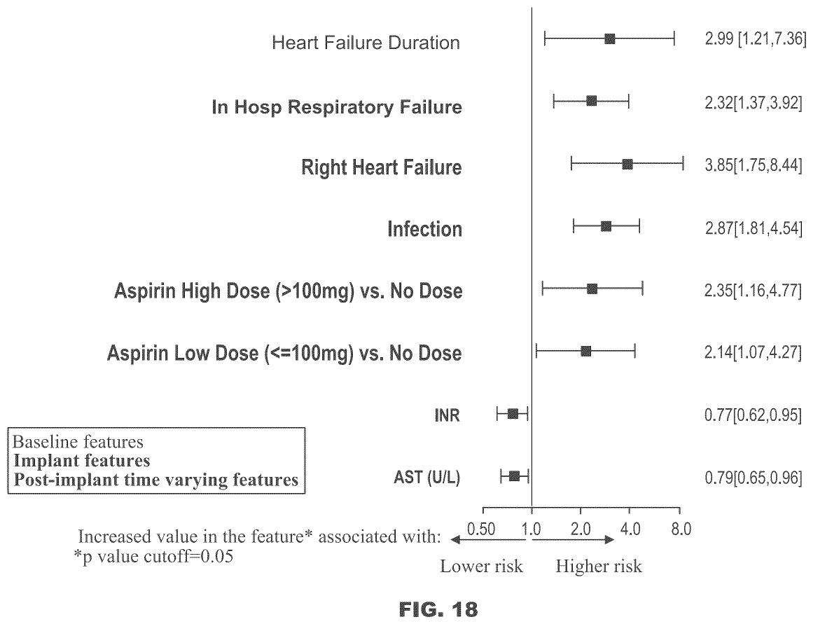

With reference to FIG. 16, FIG. 17, and FIG. 18, exemplary features used in

generation of a risk prediction for each of the states for which a risk prediction is generated and output are shown. Thus, the figures show features used in generating a risk prediction for GI bleeding, stroke, and death. These features were selected based on accuracy (adjudicated and verified data was used instead of raw data), availability (a missing rate of less than 25%), and meaningfulness. FIG. 16 depicts features associated with GI bleeding, which features have a p-value cutoff of p-value=0.05. These features include pre-implant features of: neurological-seizure history, stroke history, age, and mean pulmonary arterial pressure. These features include the peri-implant feature of in hospital GI bleeding. These features include the post-implant features of aspirin dose (low dose (<=100 mg), high dose (>100 mg), no dose), BUN (mg/dL), hemoglobin (g/dL), platelets (x10^3/uL), NTProBNP (pg/mL), total protein, and pump pulsatility index.

FIG. 17 depicts features associated with mortality, which features have a p-value cutoff of p-value=0.05. These features include pre-implant features of: significant peripheral vascular disease history, whether the patient receive(d) epinephrine, transient ischemic attack, prior valve replacement or repair, gastrointestinal history, left ventricular end-diastolic diameter (LVEDD), and/or left ventricular internal diameter end diastole and end systole (LVIDD). These features include the peri-implant feature of in hospital respiratory failure. These features include the post-implant features of right heart failure, infection, heart failure hospitalization, NTProBNP (pg/mL), BUN (mg/dL), pump speed, aspirin low dose (<=100 mg) vs. no dose, diuretic medications, albumin (g/dl), pump power, and/or total protein.

FIG. 18 depicts features associated with stroke, which features have a p-value cutoff of p-value=0.05. These features include pre-implant features of heart failure duration. These features include the peri-implant feature of in hospital respiratory failure. These features include the post-implant features of right heart failure, infection, aspirin low dose (<=100 mg) vs. no dose, aspirin high dose (>100 mg) vs. no dose, INR, and/or aspartate transferase (“AST”) (U/L).

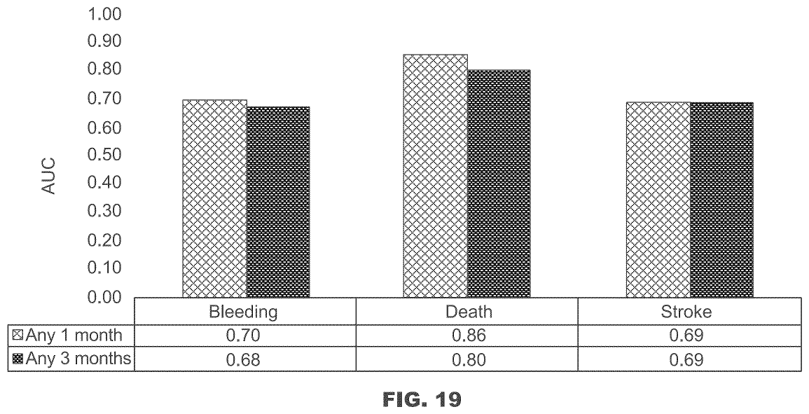

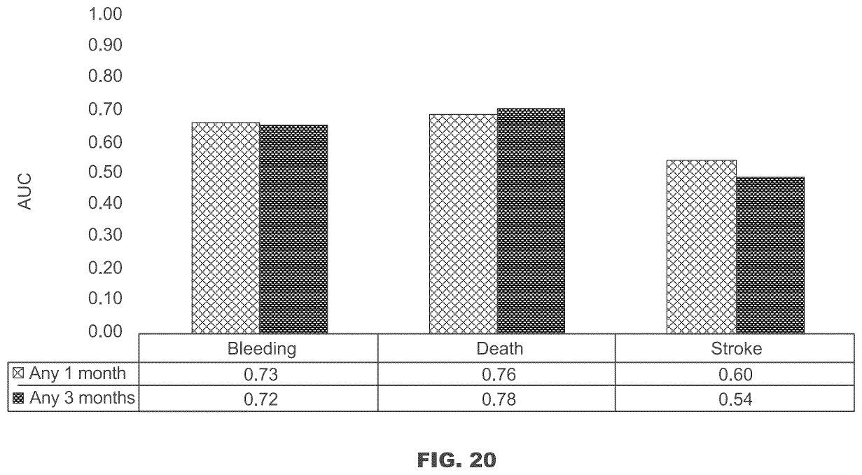

With reference to FIG. 19 and FIG. 20, results of risk predictions by the patient risk estimator 350 are shown. Specifically, FIG. 19 shows model performance at any day after discharge after a VAD implant, and the accuracy of the risk prediction of GI bleeding, stroke, and/or death in the next one month or the next three months. These results were generated using the 5-fold cross-validation on derivation data. As seen, the cross-validated AUC at one month in the derivation cohort was 0.70, 0.86, and 0.69 for GI Bleeding, death, and stroke respectively.

FIG. 20 shows model performance at any day after discharge after a VAD implant, and the accuracy of the risk prediction of GI bleeding, stroke, and/or death in the next one month or the next three months. These results were generated using the validation dataset. As seen, AUC at one month in the validation dataset was 0.73, 0.76, and 0.60 for GI Bleeding, death, and stroke respectively.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. The term “connected” is to be construed as partly or wholly contained within, attached to, or joined together, even if there is something intervening. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate embodiments of the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

1. A method of post-operative risk mitigation in patients with an implanted Ventricular Assist Device (VAD), the method comprising:
receiving a plurality of features, each of the features relating to an attribute associated with the patient having received the implanted VAD;
inputting at least some of the features into a multistate model, the multistate model comprising a plurality of states including at least one of stroke, death, and gastrointestinal bleeding, each of the states corresponding to a patient condition;
generating with the multistate model a daily prediction of a likelihood of the patient developing at least one of the conditions corresponding to the plurality of states in a predetermined time period; and
outputting on a user interface an indicator of the likelihood of the patient developing the at least one of the conditions in the predetermined time period so as to manage post-operative risk from VAD implantation.

2. The method of claim 1, wherein generating with the multistate model the daily prediction of the likelihood of the patient developing at least one of the conditions corresponding to the plurality of states in the predetermined time period comprises generating with the multistate model the daily prediction of the likelihood of the patient developing a first set of conditions corresponding to a first set of states;
and wherein outputting the indicator of the likelihood of the patient developing the at least one of the conditions in the predetermined time period comprises outputting the indicator of the likelihood of the patient developing a second set of conditions corresponding to a second set of states in the predetermined time period.

3. The method of claim 2, wherein the first set of conditions corresponding to the first set of states is greater than the second set of conditions corresponding to the second set of states.

4. The method of claim 2, wherein the first set of conditions corresponding to the first set of states is less than the second set of conditions corresponding to the second set of states.

5. The method of claim 1, wherein the multistate model generates the daily prediction for conditions corresponding to a greater number of states than are output via the indicator of the user interface.

6. The method of claim 1, wherein the plurality of features comprise at least: a baseline feature indicating a pre-operative condition of the patient; an implantation feature indicating a condition of the patient during implantation of the VAD; and a post-implantation feature indicative of a post-operative condition of the patient.

7. The method of claim 6, wherein the post-implantation feature is time-varying.

8. The method of claim 7, wherein the post-implantation feature identifies an operating attribute of the VAD.

9. The method of claim 8, wherein the operating attribute of the VAD comprises at least one of: a pump pulsatility index; a pump power; and a pump speed.

10. The method of claim 1, further comprising generating a control signal affecting an operating attribute of the VAD.

11. The method of claim 10, wherein the operating attribute of the VAD comprises at least one of: a pump pulsatility index; a pump power; and a pump speed.

12. The method of claim 1, wherein the daily prediction is generated each day for a plurality of days.

13. The method of claim 12, further comprising outputting with the user interface at least one trendline based on an aggregate of generated daily predictions.

14. The method of claim 13, wherein the at least one trendline comprises one trendline associated with one of the at least one of the conditions corresponding to one of the plurality of states.

15. The method of claim 14, wherein the one trendline indicates a change in risk with respect to time for the one of the at least one of the conditions corresponding to one of the plurality of states associated with the one trendline.

16. The method of claim 13, further comprising:
determining a change in one of the at least one trendline, the change indicating an increased risk; and
generating an alert based on the change in the one of the at least one trendline.

17. The method of claim 16, further comprising generating a modified treatment regime based on the change in the one of the at least one trendline.

18. The method of claim 13, further comprising:
determining a change in one of the at least one trendline, the change indicating a decreased risk; and
generating an alert based on the change in the trendline.

19. The method of claim 1, further comprising:
determining that at least some of the received plurality of features are aged; and
generating replacement features for at least some of the aged plurality of features; and
wherein the features inputted into the multistate model include the replacement features and the non-aged features.

20. The method of claim 19, wherein generating replacement features comprises:
selecting a replacement model for generating the replacement features; and
generating replacement features with the replacement model.

21. The method of claim 20, wherein the replacement model comprises a patient cohort selected based on a commonality of at least one attribute with the patient having received the implanted VAD.

22. The method of claim 20, wherein the replacement model selects a most recent value for use as replacement data.

23. The method of claim 1, further comprising:
determining that at least some of the received plurality of features are aged; and
determining to generate new features replacing at least one of the aged plurality of features; and
scheduling an action to generate the new features.

24. The method of claim 23, wherein the daily prediction of the likelihood of the patient developing the at least one of the conditions corresponding to the plurality of states is generated when the new features is generated.

25. The method of claim 1, wherein the multistate model utilizes logistic regression to estimate transition probability.

26.-51. (canceled)
